(19) United States
(12) Patent Application Publication
(10) Pub. No.: US 2025/0372202 A1
(43) Pub. Date: Dec. 4, 2025

(54) LEARNING INTERDEPENDENT BIOMARKERS OF DISEASE PROGRESSION FOR MEDICAL DECISION MAKING

(71) Applicant: NEC Laboratories America, Inc., Princeton, NJ (US)

(72) Inventors: Jonathan Warrell, Princeton, NJ (US); Renqiang Min, Princeton, NJ (US)

(21) Appl. No.: 19/219,271

(22) Filed: May 27, 2025

Related U.S. Application Data
(60) Provisional application No. 63/652,316, filed on May 28, 2024.
(60) Provisional application No. 63/687,434, filed on Aug. 27, 2024.

(51) Int. Cl. G16B 25/10; G06N 3/047; G16B 40/20; G16H 50/20

(52) U.S. Cl. CPC G16B 25/10; G06N 3/047; G16B 40/20; G16H 50/20

(57) ABSTRACT

Methods and systems for patient stratification include learning interdependent biomarkers as integrated time-series machine learning models. A disease stage is identified for a patient based on collected biomarker data. A treatment for the patient is performed based on the identified disease stage and a predicted future response of the patient.

### RELATED APPLICATION INFORMATION

This application claims priority to U.S. Patent Application No. 63/652,316, filed on May 28, 2024, and to U.S. Patent Application No. 63/687,434, filed on Aug. 27, 2024, each incorporated herein by reference in its entirety.

### BACKGROUND

### Technical Field

The present invention relates to time series prediction and, more particularly, to the use of biomarker data to diagnose disease.

### Description of the Related Art

Identifying and predicting particular biomarkers can help diagnose specific tumor types. Biomarkers have been identified independently for different outcomes and response types in a generalized linear model, producing either a binary output or a positive-valued continuous output.

### SUMMARY

A method for patient stratification includes learning interdependent biomarkers as integrated time-series machine learning models. A disease stage is identified for a patient based on collected biomarker data. A treatment for the patient is performed based on the identified disease stage and a predicted future response of the patient.

A system for patient stratification includes a hardware processor and a memory that stores a computer program. When executed by the hardware processor, the computer program causes the hardware processor to learn interdependent biomarkers as integrated time-series machine learning models, to identify a disease stage for a patient based on collected biomarker data, and to perform a treatment for the patient based on the identified disease stage and a predicted future response of the patient.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram of a method for patient stratification, in accordance with an embodiment of the present invention;

FIG. 2 is a block/flow diagram of a method for learning disease stages, in accordance with an embodiment of the present invention;

FIG. 3 is a block/flow diagram of a method for training a robust sparse model, in accordance with an embodiment of the present invention;

FIG. 4 is a block/flow diagram of a method for training and using a patient stratification model, in accordance with an embodiment of the present invention;

FIG. 5 is a block diagram of a healthcare facility that uses patient stratification to assist with medical decision making, in accordance with an embodiment of the present invention;

FIG. 6 is a block diagram of a computing device that can be used to learn disease stages and perform patient stratification, in accordance with an embodiment of the present invention;

FIG. 7 is a diagram of an exemplary neural network model that can be used to implement part of a machine learning model, in accordance with an embodiment of the present invention; and

FIG. 8 is a diagram of an exemplary deep neural network model that can be used to implement part of a machine learning model, in accordance with an embodiment of the present invention.

### DETAILED DESCRIPTION OF PREFERRED EMBODIMENTS

Patient responses to treatment can be predicted, for example gauging the efficacy of an immune checkpoint inhibitor in a cancer treatment. These predictions can be performed using genomics data and other covariates, such as disease type. Predicting patient response can aid in patient stratification to determine whether a given treatment is appropriate for a particular patient.

To that end, microRNA (miRNA), transcriptomics, and/or proteomics genomics data, which are convenient biomarkers, can be measured non-invasively from blood samples. Blood-based omics data can be used to help with long-term follow-up with patients, post-treatment. This helps to predict a range of outcomes, including partial and overall response, overall survival at multiple time points, and progressive versus stable disease outcomes.

These data may indicate clinical end-points, but biomarkers are often optimized for one end-point only, although they may have predictive power for other end-points. A predictor may be learned for all such end-points, and intermediate disease stages, using information for all end-points during training. As a result, separate biomarkers need not be trained for each stage, which helps if an existing biomarker is adapted to evaluate a more appropriate end-point on a new cohort. The accuracy of predicting rare outcomes may be increased by joint training with more common outcomes. In addition, previously unknown disease stages may be discovered by the model.

The present embodiments may be directed to a known-disease-stage setting and to an unknown-disease-stage setting. In a known-disease-stage setting, a predefined set of disease stage labels are known and can be assigned to patients across a series of visits where genomics and label data are collected. In an unknown-disease-stage setting, the set of disease stage labels is not known, so visits only collect genomics data. The unknown-disease-stage setting may also apply to exploratory analysis, where no treatments are given and the goal is to understand the progression of a disease before it becomes malignant, or when only survival data is available such that the only information is whether the patient lived after a treatment. Treatment effectiveness information may not be available.

Data may be collected from a patient over a series of visits, separated by a time interval. Each visit may be identified as being pre-treatment or post-treatment. If the visits occur at irregular intervals, or some are missing from the medical records, a latent variable approach may be used to register patient data to a common model architecture. Once a training data set is created, a predictive model may be learned that allows one to estimate the probability that a given patient with particular genomics data will reach a certain disease stage at a future time.

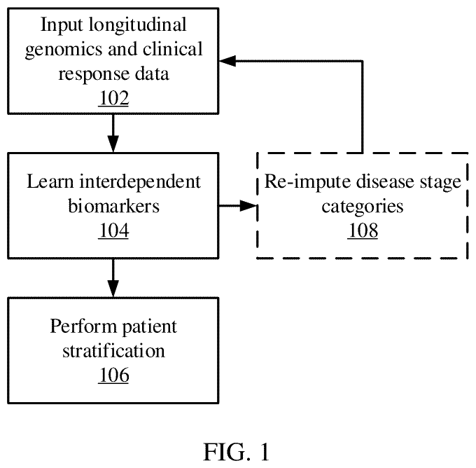

Referring now to FIG. 1, a method for patient stratification is shown. Block 102 inputs genomic and clinical response data from one or more patients. Block 104 learns interdependent biomarkers as an integrated time-series model to predict disease stage progression using annotated disease stage categories. Block 106 then performs patient stratification using the best performing derived signature and threshold for a disease type and stage. Optionally, block 108 may re-impute new disease stage categories.

Here, a biomarker may be interpreted as a predictive model of disease stage occurring at a given follow-up time learned from genomics data. For example, a subset of blood miRNA measurements and their coefficients may form a bespoke biomarker for predicting the survival of a patient given a particular treatment after predetermined period of time, via a generalized linear model. Some embodiments learn interdependent biomarkers using generalized linear predictors to model transitions between disease stages. Some embodiments may use a deep neural network architectures such as a recurrent neural network (RNN) or transformer-based architecture with causal attention.

Interdependent disease stage biomarkers for known stages may be represented as a time series, with time steps T={1, . . . , tₘₐₓ}, partitioned into sets Tₚᵣₑ and Tₚₒₛ for pre-treatment and post-treatment measurements. These sets may be assumed to be a disjoint, connected partition of T. During training, label and genomics data may be designated as Xᵢₜ and Yᵢₜ, respectively, for individual i at time t. Xᵢₜ may be a one-hot encoding vector and Yᵢₜ may be a real vector representing the genomics measurements and any additional covariates, such as a vector of miRNA expression values, age, and sex, representing he latter by a binary indicator.

The label-based prediction model may be trained by optimizing a regularized Markov chain loss:

$$L ( β \vert X , Y ) = {\sum }_{i} log {P}_{0} ( {X}_{i 0} \vert {Y}_{i 0} , {β}_{0} ) + {\sum }_{i , t > 0} log P ( {X}_{it} \vert {X}_{i , t - 1} , {Y}_{i , t} , {β}_{f ( t )} ) + {\sum }_{l , k} ( {λ}_{1} {\vert {β}_{k l} \vert}_{1} + {λ}_{2} {‖ {β}_{k l} ‖}_{*} )$$

where β={β₀, β₁, β₂, β₃}, such that each βₖ is a matrix of size (L+M)×L, where L is the number of labels and M is the number of genomic measurements and covariates, |·|₁ and |·|_{*} are the element-wise L₁ norm, and tensor nuclear norm respectively, and f, P(.|.,.,.) and P₀(. |.,.) are defined as:

$$f ( t ) = \{ \begin{matrix}1 , & if t ∈ {T}_{pre} and t - 1 ∈ {T}_{pre} \\ 2 , & if t ∈ {T}_{pos} and t - 1 ∈ {T}_{pre} \\ 3 , & if t ∈ {T}_{pos} and t - 1 ∈ {T}_{pos}\end{matrix}$$

$$P ( {X}^{′} \vert X , Y , β ) = \frac{exp ( [ {X}^{T} , {Y}^{T} ] \cdot {β}_{l ( {X}^{′} )} )}{{\sum }_{{l}^{′}} exp ( [ {X}^{T} , {Y}^{T} ] \cdot {β}_{{l}^{′}} )}$$

$${P}_{0} ( X \vert Y , β ) = \frac{exp ( [ {0}_{L}^{T} , {Y}^{T} ] \cdot {β}_{l ( X )} )}{{\sum }_{{l}^{′}} exp ( [ {0}_{L}^{T} , {Y}^{T} ] \cdot {β}_{{l}^{′}} )}$$

where l(X) denotes the label/for which Xₗ=1 and [,] denotes a horizontal concatenation operation. Each matrix βₖ represents a Markov transition kernel between disease stages conditioned on genomics measurements, and the notation βₖ t denotes the l^{th} column of βₖ, with the coefficients corresponding to output label l. Further, λ₁ and λ₂ are hyperparameters representing the weights placed on the sparsity and shrinkage regularizers, and can be set by cross-validation on a training cohort.

Since the model above is convex, it may be trained using gradient descent. Higher-order sparse features may also be included in Y (for instance, products of the expression of pairs of miRNAs). In this case, an additional L₁ norm regularizer may be placed on a low-rank representation of the higher-order features, and an extension of a factorization-based sparse learning framework. Further, there may be restrictions on the transition function P(.|.,.,.) such that certain disease stages may not follow others. For example, no disease stage may follow death. In this case, the βₗ_{_{1,}}ₗ_{₂} value may be fixed to -∞ throughout training, where l₂ cannot follow l₁. Finally, for robust training, a multi-split and step-down approach may be used for robust sparse model training, as discussed below.

The genomics prediction model may be trained similarly to the label-prediction model, using the following loss:

$$L ( γ \vert X , {Y}_{g e n} ) = {\sum }_{i , t > 0} Log P ( {( {Y}_{g e n} )}_{it} \vert {Y}_{i , t - 1} , {X}_{i , t - 1} , {γ}_{f ( t )} )$$

where γ={γ₁, γ₂, γ₃}, such that each γₖ is a matrix of size (L+M)×M_{gen} representing a Markov transition kernel between genomics measurements conditioned on disease stage, writing M_{gen} for the number of genomics measurements and Y_{gen} for the restriction of Y to the genomics measurements, and:

$$P ( {Y}_{g e n}^{′} \vert X , Y , γ ) = 𝒩 ( {Y}_{g e n}^{′} \vert γ [ X ; Y ] , σ {I}_{{M}_{g e n}} )$$

where [symbol] (·|μ, Σ) is a multi-variate normal distribution with mean u and covariance Σ, I is the identity matrix, and σ is a hyperparameter. As above, the model is convex, and may be trained using gradient descent.

At test time, either an individual patient or a validation cohort of patients with survival data is given. For an individual patient, it can be assumed that their genomics are observed at time t=0. To predict the probability that they have label l* at time t* by evaluating:

$$P ( {X}_{{t}^{*} , {l}^{*}} \vert {Y}_{0} ) = {\int }_{{Y}_{t > 0}} {\sum }_{{X}_{t < {t}^{*}}} {\prod }_{i} {P}_{0} ( {X}_{0} \vert {Y}_{0} , {β}_{0} ) {\prod }_{i , t > 0} P ( {Y}_{t} \vert {Y}_{t - 1} , {X}_{t - 1} , {γ}_{f ( t )} ) {\prod }_{i , t > 0} P ( {X}_{t} \vert {X}_{t - 1} , {Y}_{t} , {β}_{f ( t )} )$$

The marginalization across X_{t<t*} and Y_{t>0} may be evaluated using Bayesian Network belief propagation.

For a cohort of patients with survival data, the data includes (t*ᵢ, l*ᵢ) for each patient, where t*ᵢ is the last time of follow-up for patient i, and lt is an indicator for a privileged label l* at time t*ᵢ. For example, l* may indicate that the patient died, and l*ᵢ=0 or 1 depending on whether patient i is alive or dead at t*ᵢ. Then, P(X_{t*,l*}, |Y₀) may be evaluated for each patient at t*ᵢ, generating a series of predictors p₁, p₂, . . . p_{N}, where pᵢ=P(X_{t*,l*}, |Y₀), and the performance of the model with respect to the validation cohort may be summarized by evaluating the area under the curve of this predictor with respect to the label of interest l*ᵢ.

Due to the joint training of the model with respect to multiple disease stages, the performance of the model as biomarker may be evaluated with respect to alternative end-points, corresponding to any of the labels in the model. Further, this metric provides an alternative evaluation metric to the Cox-regression Hazards Ratio of a biomarker at a predefined cut-point typically used in survival analysis. A Hazards Ratio may be calculated at a particular threshold p* for comparison by fitting a univariate Cox-regression model to the log pᵢ scores, and using the cut-point log p*.

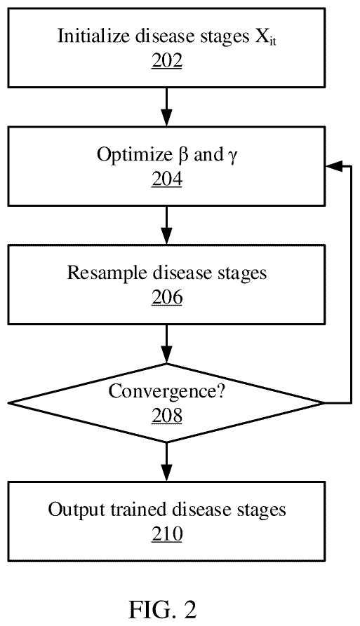

Referring now to FIG. 2, a method for estimating disease stages X is shown, if these are not known a priori. The disease stages X are estimated jointly with β and γ during training. The loss described above may be used, but training may proceed using a Monte-Carlo expectation maximization process. Block 202 initializes the disease stages Xᵢₜ by sampling from a uniform distribution over labels. Since the labels are treated as unknown, only the total number of labels, L is needed as a hyperparameter, which may be set by cross-validation using the log-likelihood on hold-out subjects across multiple data splits. Each label is represented by a one-hot vector of length L, and is sampled with probability 1/L.

Block 204 optimizes B and y using the loss functions L(γ|X, Y_{gen}) and P(X_{t*,l*}, |Y₀) holding X fixed. This optimization may be performed using gradient descent or the robust multi-split approach described below. Block 206 re-samples the disease stages Xᵢₜ by sampling from:

$$P ( X \vert Y , β ) = {\prod }_{i} {P}_{0} ( {X}_{i 0} \vert {Y}_{i 0} , {β}_{0} ) {\prod }_{i , t > 0} P ( {Y}_{it} \vert {Y}_{i , t - 1} , {X}_{i , t - 1} , {γ}_{f ( t )} ) {\prod }_{i , t > 0} P ( {X}_{it} \vert {X}_{i , t - 1} , {Y}_{i , t} , {β}_{f ( t )} )$$

using the current estimates for β and γ.

Block 208 determines whether the training has reached a convergence criterion, such as determining whether the maximal change in β or γ is below a threshold τ. If the process has not converged, it returns to block 204 to update β and γ. If the process has converged, then block 210 outputs the learned disease stages Xᵢₜ.

For the case where only some disease stages are known while others are unknown, such as in a pretreatment stage or death, these known stages may be fixed throughout the stage learning process. The sampling in block 206 may omit these known labels. Block 206 may be decomposed as a set of independent Markov-chains (one per patient), the disease stages may be sampled by applying the Viterbi algorithm.

If time-points are missing for certain patients during training, or the time-points are not regularly spaced, this training may be adapted. For missing time-points, the label and genomics data at these time points may be marginalized across, using belief propagation. For irregularly spaced time-points, the lowest common time-unit may be used (for example, a day or a week, depending on the maximum frequency with which visits were made), and the observation sequences padded with missing observations for all visits that were separated by longer than this lowest common time-unit, which are marginalizes across using P(X_{t*,l*}, |Y₀) with belief propagation.

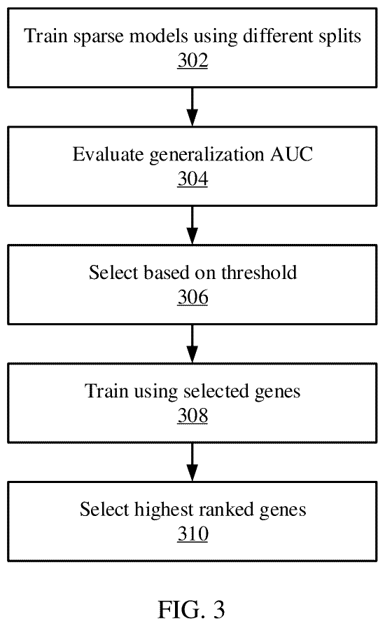

Referring now to FIG. 3, training of a robust sparse model is shown. A large number of sparse models may be trained in block 302, each using a different split of the training data. Their generalization area under a receiver operating characteristic (ROC) curve may be evaluated on the internal test split in block 304.

Block 306 sets a threshold on this area under the curve, including all genes (or miRNAs/proteins depending on the type of genomics) from any of the sparse models achieving an area under the curve that is above this threshold. Block 308 then trains a model including only such genes, without the sparsity penalty, on all the training data. The genes are then removed one by one in order of their absolute β coefficients (from smallest to largest), until a desired number of genes is included. For example, a biomarker may have a maximum size, Mₘₐₓ.

An initial example using a least absolute shrinkage and selection operator (LASSO) based predictor is described below, which does not include disease stage information (e.g., a single generalized linear model), before describing how the same method is applied to a set of interdependent disease stage biomarkers as introduced above. A set of m=1 . . . 300 logistic regression (LR) models may be trained with a least absolute shrinkage and selection operator LASSO penalty to predict R. For each model, the samples are divided into an 80/20 training/testing split. The terms “training” and “testing” refer to subsets of the discovery cohort.

The multiple random splits introduce variability that ensures the robustness of the resultant gene signatures by mitigating overfitting to any specific data partition. The validation cohort remains untouched throughout the training, so that the validation cohort provides an unbiased estimate of the generalization of the signatures.

For each training split, genes are selected that are significantly differentially expressed on the training partition at False Discovery Rate (FDR)<0.2 to allow the LASSO To choose from a sufficient number of genes. This set of genes is used to optimize a LASSO model, for example using ten-fold cross-validation to find a A penalty starting from 100 random seeds. The random seeds are used to ensure the robustness of the models for each of the 80/20 splits to different initialization conditions. Thus, for a given data split, sets of coefficients are trained such that β_{s,j} is the coefficient of gene j in the model using seed s.

Genes are selected that have a non-zero coefficient for at least one of the seeds. To get the final coefficients for a model m, the LR model may be refit without the λ penalty using only those selected genes. The coefficients

$${β}_{m , j}^{f i n a l}$$

for a model m is thereby produced, so that the final LASSO score for patient i in the model m is

$${s}_{m , i} = {\sum }_{j} {β}_{m , j}^{f i n a l} {M}_{ij} ,$$

where

Mᵢⱼ is the expression of the j^{th} genomics feature in the i^{th} individual.

To create a final compartment signature based on the trained LASSO models, AUCₘ is calculated as the area under the curve of each model m. Those models for which AUCₘ is greater than a threshold value (e.g., 0.7) are selected. The final signature then uses all genes occurring in at least t models: Σₛ[(β_{s,j}≠0]≥t, where t is set to the largest value such that the number of selected genes is 30.

An intermediate signature may be calculated with a larger number of genes than required (e.g. thirty genes), again by retraining an LR model without the λ penalty using only those selected genes, including all patients in the original cohort. This produces intermediate signature score

$${s}_{i}^{inter} = {\sum }_{j} {β}_{j}^{inter} {M}_{i j}$$

patient i. To calculate a final signature, a step-down process may be used to compactify the intermediate signature so that the resulting signature has fewer than ten genes.

The coefficients may be ordered by their absolute size (e.g., |β₁|≥|β₂| . . . ). For a compact signature of size j′, the score has the value

$${s}_{i}^{{j}^{′}} = {\sum }_{j = 1 … {j}^{′}} {β}_{j}^{inter} {M}_{i j} .$$

To find the final signature, the j′≤10 are selected with the best accuracy on the discovery cohort, j*, such that

$${s}_{i}^{f i n a l} = {s}_{i}^{{j}^{*}} .$$

Hence, evaluating the final signature score sums the expression values across the reduced set of genes, weighted by their final coefficients. The final signature may be described as a logistic regression model trained on LASSO-selected genes, with the lowest absolute coefficients pruned.

In the case of a set of interdependent biomarkers, the above method for learning a robust sparse model may be used to select a small set of genes (or other genomics features) to include in the model across all disease stages. Hence, all genes with non-zero coefficients in any column of the matrices β₀, β₁, β₂, β₃ are selected in block 308, and the step-down procedure in block 310 orders the genes by their maximum absolute coefficient in any column of the same matrices.

Cell type-specific signatures may be developed for both tumor and stromal compartments in digital spatial profiling (DSP) data, using a disease stage model with unknown stages to predict adjuvant lung-cancer patients response to anti PD-L₁ treatment, based on the split-sample-based method above. Matrices

$${M}_{i j}^{tumor} and {M}_{i j}^{stroma}$$

include cell fractions for each patient i and cell type j in the respective compartments prior to treatment. If there are 14 distinct cell types classified across tumor and stromal compartments, then M_{gen}=14 for the number of genomics features. For each patient, Progression Free Survival (PFS) data may be used as the focal label for training, including time in months and an event indicator: 1 for disease progression/death and 0 for alive/progression-free.

Compartment-specific gene signatures may be developed based on cell type models using a disease stage model with unknown stages to predict adjuvant lung-cancer patients response to anti PD-L₁ treatment, based on a split-sample-based method above. The gene signature matrix may be obtained from an analytical tool such as CIBERSORTx, which provides reference gene expression profiles for various cell types. Additionally, a gene list may be obtained from CD4 T cells, macrophages, granulocytes, and malignant cells based on the public lung cancer scRNA-seq dataset Lung Cancer Atlas (LuCA). This may be accomplished using the R package mastR. Briefly using mastR, 892,296 cells from LuCA were aggregated into pseudo samples based on the sample and cell type, resulting in 2215 pseudo-samples each with >50 cells each for cell type identification (a total of 14 cell types were identified). The aggregated pseudobulked samples were used to generate refined markers for the selected cell types using a differential expression and ranked product permutation test workflow implemented by mastR, with default parameters used for all mastR analyses. Matrices

$${M}_{i j}^{tumor} and {M}_{i j}^{stroma} ,$$

containing gene expression counts for each patient i and gene j, were obtained in the respective compartments. For each patient, PFS data may be used as the focal label for training, with time in months and an event indicator: 1 for disease progression/death and 0 for alive/progression-free.

A biomarker may be developed for non-small-cell lung cancer (NSCLC) to predict a patient's adjuvant response to immune checkpoint inhibitor and chemotherapy treatment, by drawing blood samples from patients at a series of 10 time points (each a month apart), and using a Somalogic microarray to read the expression of 20 miRNAs to provide the genomics measurements at each time-point. miRNA-base signatures may be trained using a disease stage model with known disease labels as above. Disease stage labels may be assigned by an oncologist at each time point to each patient: PT (pre-treatment), PR (partial response), SD (stable disease), PD (progressive disease), DE (Death). The model may be constrained so that no stage may precede PT, and no stage may follow DE.

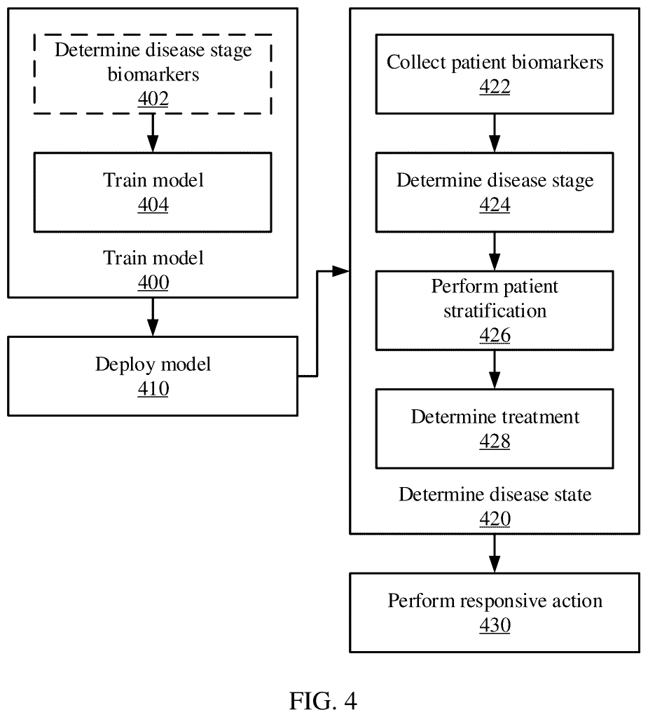

Referring now to FIG. 4, a method for training and using a model for patient stratification based on biomarkers is shown. Block 400 trains the model, which includes determining disease stage biomarkers 402 and training a model 404 to identify disease stages. The determination of disease stage biomarkers 402 is an optional step that depends on the target disease trajectory, where disease stages may already be known. Block 410 then deploys the trained model, for example by copying parameters of the model to a target system where new biomarker data is available. In some cases, where training and inference are performed by the same system, deployment 410 may be omitted.

Block 420 determines a disease stage for a given patient. Block 422 collects biomarker information from the patient, for example using test results to identify miRNA, transcriptomics, and/or proteomics genomics data. Block 424 uses the trained model to identify disease stage using the collected biomarkers. This disease stage is used to perform patient stratification in block 426, which can be used to select an appropriate treatment in block 428. Block 430 then performs an appropriate action responsive to the identified disease stage, such as automatically administering the determined treatment.

To select an appropriate treatment for a patient, multiple models may be trained for different types of treatment and/or treatment combinations, for instance, different types of chemotherapy and immunotherapy. For a new patient, the disease stage is determined for each model using available genomics data. Then, the expected overall survival (OS) time or progression free survival (PFS) time is evaluated for each treatment, or the probability of overall survival or PFS at a key follow-up time (e.g. 2 years) is evaluated, and the treatment which maximizes either of these metrics is chosen. Alternatively, in choosing whether or not to administer a given treatment, either of the metrics above may be evaluated for a new patient given the specific treatment, and the treatment only administered if the metric exceeds a predefined threshold (chosen according to a predefined criterion, for example, the patient is expected to have OS or PFS survival exceeding 2 years, or the probability of stable disease for at least a year following the treatment exceeds 0.9).

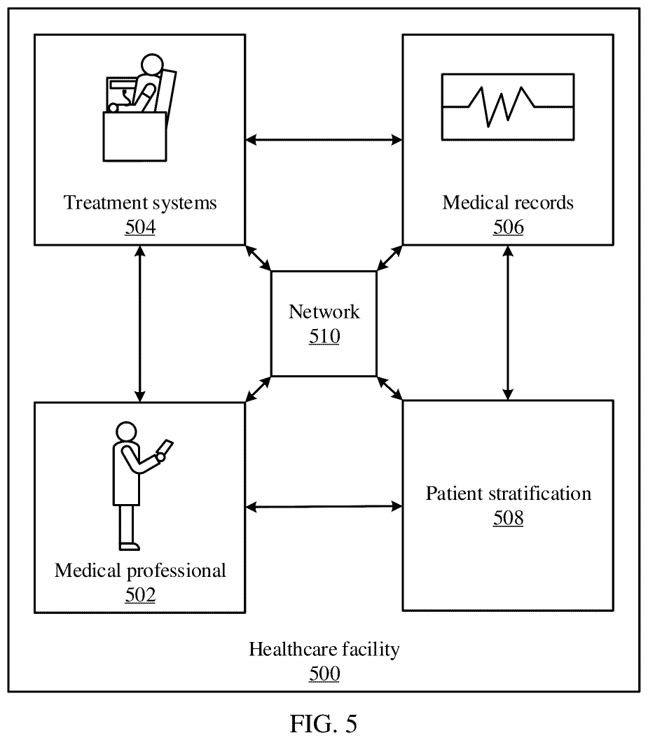

Referring now to FIG. 5, a diagram of time series analysis is shown in the context of a healthcare facility 500. Patient stratification 508 may be performed using a trained model, for example by inputting biomarker data for a patient based on tests and their medical records 506.

The healthcare facility may include one or more medical professionals 502 who review information extracted from a patient's medical records 506 to determine their healthcare and treatment needs. These medical records 506 may include self-reported information from the patient, test results, and notes by healthcare personnel made to the patient's file. Treatment systems 504 may furthermore monitor patient status to generate medical records 506 and may be designed to automatically administer and adjust treatments as needed.

Based on information drawn from the patient stratification 508, the medical professionals 502 may then make medical decisions about patient healthcare suited to the patient's needs. For example, the medical professionals 502 may make a diagnosis of the patient's health condition and may prescribe particular medications, surgeries, and/or therapies that are appropriate to the stage of a disease.

The different elements of the healthcare facility 500 may communicate with one another via a network 510, for example using any appropriate wired or wireless communications protocol and medium. Thus patient stratification 508 receives data from treatment systems 504, medical professionals 502, and from medical records 506, and updates the medical records 506 with information about disease stage. The patient stratification 508 may further coordinate with treatment systems 504 in some cases to automatically administer or alter a treatment. For example, patient stratification 508 may indicate an advanced stage of a disease, which can cause the treatment systems 504 to automatically start, alter, or halt the administration of the treatment.

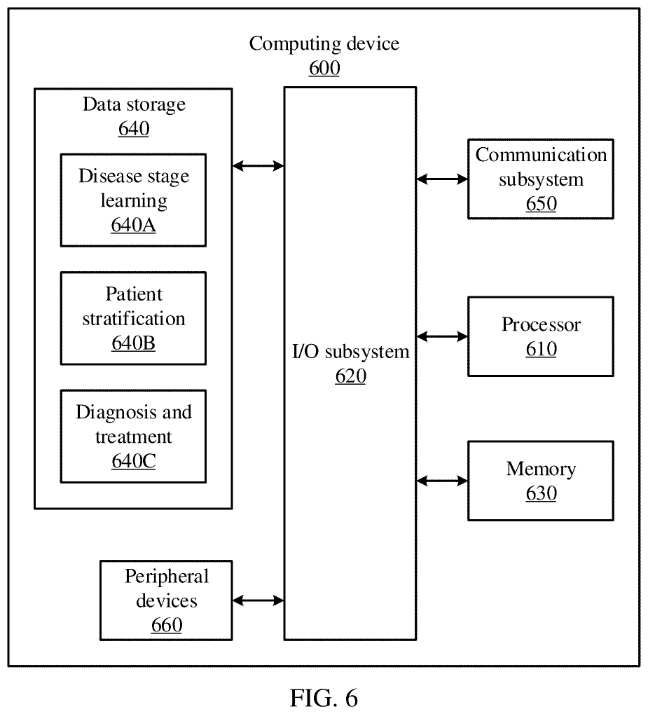

Referring now to FIG. 6, an exemplary computing device 600 is shown, in accordance with an embodiment of the present invention. The computing device 600 is configured to perform visual question answering.

The computing device 600 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 600 may be embodied as one or more compute sleds, memory sleds, or other racks, sleds, computing chassis, or other components of a physically disaggregated computing device.

As shown in FIG. 6, the computing device 600 illustratively includes the processor 610, an input/output subsystem 620, a memory 630, a data storage device 640, and a communication subsystem 650, and/or other components and devices commonly found in a server or similar computing device. The computing device 600 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 630, or portions thereof, may be incorporated in the processor 610 in some embodiments.

The processor 610 may be embodied as any type of processor capable of performing the functions described herein. The processor 610 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 630 may be embodied as any type of volatile or non-volatile memory or data storage capable of performing the functions described herein. In operation, the memory 630 may store various data and software used during operation of the computing device 600, such as operating systems, applications, programs, libraries, and drivers. The memory 630 is communicatively coupled to the processor 610 via the I/O subsystem 620, which may be embodied as circuitry and/or components to facilitate input/output operations with the processor 610, the memory 630, and other components of the computing device 600. For example, the I/O subsystem 620 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 620 may form a portion of a system-on-a-chip (SOC) and be incorporated, along with the processor 610, the memory 630, and other components of the computing device 600, on a single integrated circuit chip.

The data storage device 640 may be embodied as any type of device or devices configured for short-term or long-term storage of data such as, for example, memory devices and circuits, memory cards, hard disk drives, solid state drives, or other data storage devices. The data storage device 640 can store program code 640A for learning disease stages, 640B for patient stratification, and/or 640C for performing diagnosis and treatment. Any or all of these program code blocks may be included in a given computing system. The communication subsystem 650 of the computing device 600 may be embodied as any network interface controller or other communication circuit, device, or collection thereof, capable of enabling communications between the computing device 600 and other remote devices over a network. The communication subsystem 650 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 600 may also include one or more peripheral devices 660. The peripheral devices 660 may include any number of additional input/output devices, interface devices, and/or other peripheral devices. For example, in some embodiments, the peripheral devices 660 may include a display, touch screen, graphics circuitry, keyboard, mouse, speaker system, microphone, network interface, and/or other input/output devices, interface devices, and/or peripheral devices.

Of course, the computing device 600 may also include other elements (not shown), as readily contemplated by one of skill in the art, as well as omit certain elements.

For example, various other sensors, input devices, and/or output devices can be included in computing device 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 600 are readily contemplated by one of ordinary skill in the art given the teachings of the present invention provided herein.

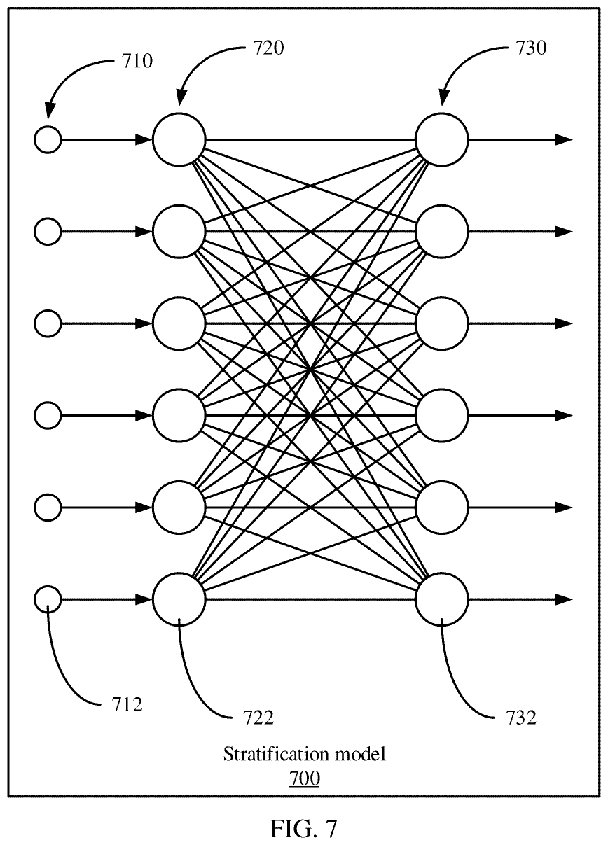

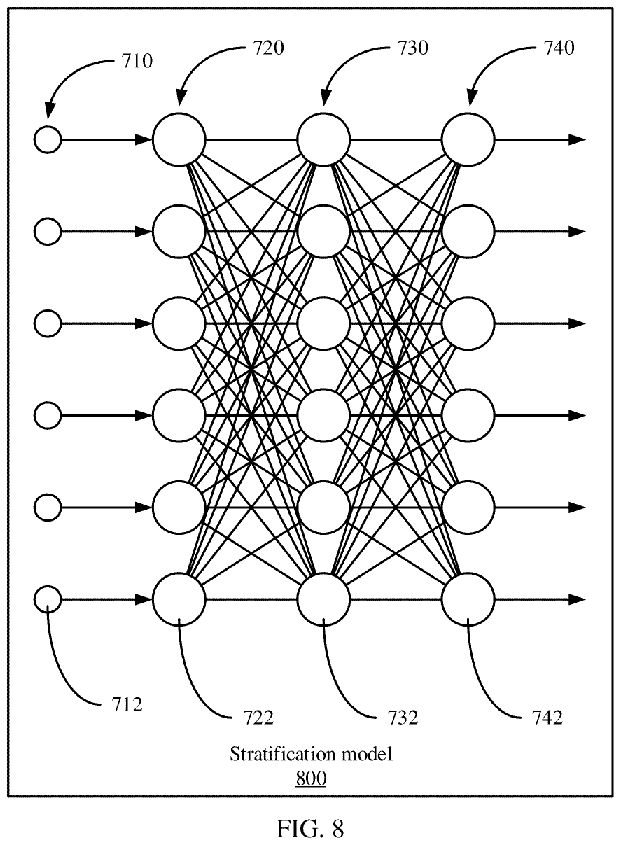

Referring now to FIGS. 7 and 8, exemplary neural network architectures are shown, which may be used to implement parts of the present machine learning models, such as the stratification model 700/800, for the case that a Recurrent Neural Network (RNN) or transformer with causal attention is used as the underlying time series model. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 720 of source nodes 722, and a single computation layer 730 having one or more computation nodes 732 that also act as output nodes, where there is a single computation node 732 for each possible category into which the input example could be classified. An input layer 720 can have a number of source nodes 722 equal to the number of data values 712 in the input data 710. The data values 712 in the input data 710 can be represented as a column vector. Each computation node 732 in the computation layer 730 generates a linear combination of weighted values from the input data 710 fed into input nodes 720, and applies a non-linear activation function that is differentiable to the sum. The exemplary simple neural network can perform classification on linearly separable examples (e.g., patterns).

A deep neural network, such as a multilayer perceptron, can have an input layer 720 of source nodes 722, one or more computation layer(s) 730 having one or more computation nodes 732, and an output layer 740, where there is a single output node 742 for each possible category into which the input example could be classified. An input layer 720 can have a number of source nodes 722 equal to the number of data values 712 in the input data 710. The computation nodes 732 in the computation layer(s) 730 can also be referred to as hidden layers, because they are between the source nodes 722 and output node(s) 742 and are not directly observed. Each node 732, 742 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 732 in the one or more computation (hidden) layer(s) 730 perform a nonlinear transformation on the input data 712 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor-or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for patient stratification, comprising:
learning interdependent biomarkers as integrated time-series machine learning models;
identifying a disease stage for a patient based on collected biomarker data; and
performing a treatment for the patient based on the identified disease stage and a predicted future response of the patient.

2. The method of claim 1, wherein learning the biomarkers uses predetermined disease stage labels.

3. The method of claim 2, wherein learning the biomarkers includes a label-based prediction model optimized with a loss function:
$$L ( β \vert X , Y ) = {\sum }_{i} log {P}_{0} ( {X}_{i 0} \vert {Y}_{i 0} , {β}_{0} ) + {\sum }_{i , t > 0} log P ( {X}_{it} \vert {X}_{i , t - 1} , {Y}_{i , t} , {β}_{f ( t )} ) + {\sum }_{l , k} ( {λ}_{1} {\vert {β}_{k l} \vert}_{1} + {λ}_{2} {‖ {β}_{k l} ‖}_{*} )$$
where X is a set of disease stages, Y is a set of genomic data, β={β₀, β₁, β₂, β₃}, such that each βₖ is a matrix of size (L+M)×L, where L is a number of labels and M is a number of genomic measurements and covariates, |·|₁ and |·|_{*} are an element-wise L₁ norm and tensor nuclear norm respectively, P(·) and P₀(·) are probability functions, and λ₁ and λ₂ are weighting coefficients.

4. The method of claim 2, wherein learning the biomarkers includes a genomics-based prediction model optimized with a loss function:
$$L ( γ \vert X , {Y}_{g e n} ) = {\sum }_{i , t > 0} log P ( {( {Y}_{g e n} )}_{it} \vert {Y}_{i , t - 1} , {X}_{i , t - 1} , {γ}_{f ( t )} )$$
where X is a set of disease stages, Y_{gen} is a set of genomic data, γ={γ₁, γ₂, γ₃}, such that each γₖ is a matrix of size (L+M)×M_{gen}, writing M_{gen} for a number of genomics measurements and Y_{gen} for a restriction of Y to the genomics measurements, and P(·) is a probability function.

5. The method of claim 1, wherein identifying the disease stage for the patient includes a probability that the collected biomarker data has a label l* at time t:
$${\int }_{{Y}_{t > 0}} {\sum }_{{X}_{t < {t}^{*}}} {\prod }_{i} {P}_{0} ( {X}_{0} \vert {Y}_{0} , {β}_{0} ) {\prod }_{i , t > 0} P ( {Y}_{t} \vert {Y}_{t - 1} , {X}_{t - 1} , {γ}_{f ( t )} ) {\prod }_{i , t > 0} P ( {X}_{t} \vert {X}_{t - 1} , {Y}_{t} , {β}_{f ( t )} )$$
where Xₜ is a disease stage, Yₜ is biomarker information, and P(·) is a probability function.

6. The method of claim 1, wherein the biomarkers are learned using unknown disease stage labels.

7. The method of claim 6, further comprising learning the disease stage labels using an iterative sampling of disease stages and optimization.

8. The method of claim 1, wherein learning the biomarkers includes selecting biomarkers that have an area under a curve that is above a threshold value.

9. The method of claim 1, wherein learning the biomarkers uses labels for multiple clinical end-points of interest.

10. The method of claim 1, wherein the disease stage is used for patient stratification to assist in medical decision making.

11. A system for patient stratification, comprising:
a hardware processor; and
a memory that stores a computer program which, when executed by the hardware processor, causes the hardware processor to:
learn interdependent biomarkers as integrated time-series machine learning models;
identify a disease stage for a patient based on collected biomarker data; and
perform a treatment for the patient based on the identified disease stage and a predicted future response of the patient.

12. The system of claim 11, wherein the learning of the biomarkers uses predetermined disease stage labels.

13. The system of claim 12, wherein the learning of the biomarkers includes a label-based prediction model optimized with a loss function:
$$L ( β \vert X , Y ) = {\sum }_{i} log {P}_{0} ( {X}_{i 0} \vert {Y}_{i 0} , {β}_{0} ) + {\sum }_{i , t > 0} log P ( {X}_{it} \vert {X}_{i , t - 1} , {Y}_{i , t} , {β}_{f ( t )} ) + {\sum }_{l , k} ( {λ}_{1} {\vert {β}_{k l} \vert}_{1} + {λ}_{2} {‖ {β}_{k l} ‖}_{*} )$$
where X is a set of disease stages, Y is a set of genomic data, β={β₀, β₁, β₂, β₃}, such that each βₖ is a matrix of size (L+M)×L, where L is a number of labels and M is a number of genomic measurements and covariates, |·|₁ and |·|_{*} are an element-wise L₁ norm and tensor nuclear norm respectively, P(·) and P₀(·) are probability functions, and λ₁ and λ₂ are weighting coefficients.

14. The system of claim 12, wherein the learning of the biomarkers includes a genomics-based prediction model optimized with a loss function:
$$L ( γ \vert X , {Y}_{g e n} ) = {\sum }_{i , t > 0} log P ( {( {Y}_{g e n} )}_{it} \vert {Y}_{i , t - 1} , {X}_{i , t - 1} , {γ}_{f ( t )} )$$
where X is a set of disease stages, Y_{gen} is a set of genomic data, γ={γ₁, γ₂, γ₃}, such that each γₖ is a matrix of size (L+M)×M_{gen}, writing M_{gen} for a number of genomics measurements and Y_{gen} for a restriction of Y to the genomics measurements, and P(·) is a probability function.

15. The system of claim 11, wherein identification of the disease stage for the patient includes a probability that the collected biomarker data has a label l* at time t:
$${\int }_{{Y}_{t > 0}} {\sum }_{{X}_{t < {t}^{*}}} {\prod }_{i} {P}_{0} ( {X}_{0} \vert {Y}_{0} , {β}_{0} ) {\prod }_{i , t > 0} P ( {Y}_{t} \vert {Y}_{t - 1} , {X}_{t - 1} , {γ}_{f ( t )} ) {\prod }_{i , t > 0} P ( {X}_{t} \vert {X}_{t - 1} , {Y}_{t} , {β}_{f ( t )} )$$
where Xₜ is a disease stage, Yₜ is biomarker information, and P(·) is a probability function.

16. The system of claim 11, wherein the biomarkers are learned using unknown disease stage labels.

17. The system of claim 16, wherein the computer program further causes the hardware processor to learn the disease stage labels using an iterative sampling of disease stages and optimization.

18. The system of claim 11, wherein the learning of the biomarkers includes selecting biomarkers that have an area under a curve that is above a threshold value.

19. The system of claim 11, wherein the learning of the biomarkers uses labels for multiple clinical end-points of interest.

20. The system of claim 11, wherein the disease stage is used for patient stratification to assist in medical decision making.
